(19) United States
(12) Patent Application Publication
(10) Pub. No.: US 2025/0341516 A1
(43) Pub. Date: Nov. 6, 2025

(54) COMPOSITIONS FOR USE WITH MULTIPLE FLUOROPHORES AND METHODS OF USING

(71) Applicant: BioLegend, Inc., San Diego, CA (US)

(72) Inventors: Swetha Mohan, San Diego, CA (US); Anagha Ashok Divekar, San Diego, CA (US)

(21) Appl. No.: 19/184,129

(22) Filed: Apr. 21, 2025

Related U.S. Application Data
(60) Provisional application No. 63/640,930, filed on May 1, 2024.

(51) Int. Cl. G01N 33/533

(52) U.S. Cl. CPC G01N 33/533

(57) ABSTRACT

Improved compositions and methods of using the compositions in detecting the presence or absence of an analyte are disclosed. These compositions may provide improved signal with reduced background and in some aspects fewer false double positive populations observed, by dramatically reducing stacking of polymer dyes and allowing the use of multiple polymer dyes in a single tube thereby improving methods of using such compositions for fluorescent analyses.

### CROSS REFERENCE TO RELATED APPLICATIONS

This application depends from and claims priority to U.S. Provisional Application No. 63/640,930 filed May 1, 2024, the entire contents of which are incorporated herein by reference.

### FIELD

The present description relates to compounds for improving studies involving detection of a target analyte using fluorescent labeling, methods of making, and methods of using.

### BACKGROUND

Detecting, tracing and identifying biological materials are increasingly important in medical diagnoses, treatment, and research. The advancement of imaging technologies that are rapid, robust and are capable of detecting desired targets with low error are essential to these studies. Fluorescence technologies are increasingly utilized due to detection power and safety relative to other detection methodologies. The use of multiple fluorophores in a single solution for detection represents a powerful method for identification of multiple target types in a single sample.

Antibody cocktails are commonly custom made per the user's desired target collection. These cocktails commonly involve use of two or more antibodies and often over 20 different antibodies simultaneously. The numerous antibodies require individual dyes so as to be discernable from other antibodies in the system. Often the system requires the use of multiple polymer dyes as these dyes offer advantages over single dyes including the ability to tune fluorescent properties and improve signal. Polymer dyes, however, tend to stack against each other when mixed and stored together, creating unacceptably high background in various analyses and often some level of false double positive populations observed.

Prior solutions to polymer dye stacking included keeping multiple polymer conjugated antibodies separated prior to use at the site of analysis. This is inconvenient for users at least as multiple vials need to be handled, and also slows down the overall process due to increased staining time and increasing chance of error.

Thus, there is a continuing need for improved compositions that can be used to improve analyses using multiple dyes in a single tube, and methods of using such compositions.

### SUMMARY

The following summary of the disclosure is provided to facilitate an understanding of some of the innovative features unique to the disclosure and is not intended to be a full description. A full appreciation of the various aspects of the disclosure can be gained by taking the entire specification, claims, drawings, and abstract as a whole.

Improved compositions and methods of using the compositions in detecting the presence or absence of an analyte are disclosed. These compositions may provide improved signal with reduced background and optionally fewer false double positive populations observed by dramatically reducing stacking of polymer dyes and allowing the use of multiple polymer dyes in a single tube thereby improving methods of using such compositions for fluorescent analyses.

As such, provided are buffering compositions that include one or more buffering agents, one or more celluloses or derivatives thereof, the celluloses or derivatives thereof at a concentration of less than about 3 wt % of the buffering composition; and a polyethylene glycol, said polyethylene glycol present at less than or equal to about 3 wt % of the buffering composition. A cellulose is optionally a linear or branched alkyl cellulose. An alkyl cellulose is optionally a C₁-C₄ alkyl cellulose. In some aspects, a cellulose is a methyl cellulose. Optionally a cellulose is a hydroxypropyl cellulose. Optionally, a cellulose is a branched chain alkyl cellulose. In some aspects, two or more celluloses are included, optionally the cellulose is a combination of methyl cellulose and hydroxypropyl cellulose. In some aspects, a cellulose is present at about 0.1 wt % to about 3 wt %, optionally about 0.25 wt % to about 3 wt %. In any of the aspects as provided herein, a buffering agent is optionally characterized by a pKa of 6-8. Optionally, a buffering agent includes a phosphate, optionally as phosphate buffered saline. Optionally, a buffering composition further includes a sugar, an albumin, or both. Optionally, an albumin is bovine serum albumin or human serum albumin. Optionally, an albumin is present at about 0.5 wt %. In some aspects, a buffering composition includes about 0.1 wt % to 3 wt % methyl cellulose, about 0.01 wt % to 3 wt % hydroxypropyl cellulose, and about 0.1 wt % to about 3 wt % polyethylene glycol, optionally about 0.25 wt % to 3 wt % methyl cellulose and about 0.25 wt % to 3 wt % hydroxypropyl cellulose, optionally about 2.0 wt % to 3.0 wt % methyl cellulose and about 2.0 wt % to 3 wt % hydroxypropyl cellulose, and about 0.125 wt % to about 3 wt % polyethylene glycol, optionally about 2.5 wt % methyl cellulose and about 2.5 wt % hydroxypropyl cellulose, and further comprising about 0.125 wt % to about 3 wt % polyethylene glycol. A polyethylene glycol is optionally polyethylene glycol monomethylether 550, poly(ethylene glycol) 8000, or a combination thereof.

Also provided are methods of analyzing a biological sample that may include obtaining a biological sample, forming an analysis composition by contacting the biological sample with a cocktail comprising the buffer composition as described in the foregoing paragraph or otherwise herein and one or more antibodies, the one or more antibodies conjugated to one or more florescent dyes, and subjecting the analysis composition to analysis by detecting one or more of the dyes in contact with a portion of the biological sample. Optionally, the cocktail includes two or more antibodies specific for different target molecules, wherein an antibody specific for a first target molecule is conjugated to a first dye, and wherein an antibody specific for a second target molecule is conjugated to a second dye, the first dye and the second dye characterized by different fluorescent properties (e.g. absorption wavelength width, emission wavelength width, absorption maximum, emission maximum, Stokes shift, lifetime, anisotropy, photon yield, signal intensity, etc.). Optionally, the cocktail includes 2 to 23 antibodies directed to different target molecules and each conjugated to a different dye. In some aspects, at least two of the antibodies are conjugated to different brilliant violet dyes. Optionally, a dye is a polymer dye. A polymer dye is optionally Brilliant Violet 421™, Brilliant Violet 510™, Brilliant Violet 570™, Brilliant Violet 605™ Brilliant Violet 650™, Brilliant Violet 711™, Brilliant™ Violet 750, or Brilliant Violet 785™ Optionally, in place of or in addition to a polymer dye, a dye is optionally a rhodamine dye, a fluorescein dye, a coumarin dye, an indole dye, or any combination thereof. Optionally, a dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof. A method as provided herein optionally includes a cocktail including methyl cellulose, a hydroxypropyl cellulose and a polyethylene glycol, optionally about 0.1 wt % to 3 wt % methyl cellulose, about 0.01 wt % to 3 wt % hydroxypropyl cellulose, and about 0.1 wt % to about 3 wt % polyethylene glycol. Optionally, in a method as provided herein includes use of a cocktail including about 0.25 wt % to 3 wt % methyl cellulose and about 0.25 wt % to 3 wt % hydroxypropyl cellulose, and about 0.125 wt % to about 3 wt % polyethylene glycol. Optionally, a cocktail includes about 2 wt % to about 3 wt % methyl cellulose and about 2 wt % to about 3 wt % hydroxypropyl cellulose, and further including about 0.125 wt % to about 3 wt % polyethylene glycol. In any method as provided herein, the sample may be analyzed by flow cytometry, FISH, immunohistochemistry, sandwich assay, Southern blot, western blot, microarray, or substrate binding assay. Optionally, a sample may be analyzed by flow cytometry.

Also provided are methods of forming a cocktail for use in analysis of a biological sample that include combining a buffering composition as described above or as otherwise provided herein and one or more antibodies, the one or more antibodies conjugated to one or more florescent dyes. Optionally, the cocktail includes about 0.1 wt % to 3 wt % methyl cellulose, about 0.01 wt % to 3 wt % hydroxypropyl cellulose, and about 0.1 wt % to about 3 wt % polyethylene glycol. Optionally, the dyes include one or more polymer dyes, optionally Brilliant Violet 421™, Brilliant Violet 570™, Brilliant Violet 605™, Brilliant Violet 650™, Brilliant Violet 711™, or Brilliant Violet 785™. Optionally, in place of or in addition to a polymer dye, a dye may include a rhodamine dye, a fluorescein dye, a coumarin dye, an indole dye, or any combination thereof.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings.

FIG. 1A illustrates studies using flow cytometry with polymer dye-conjugated antibodies in PBS buffer revealing the presence of high background and false double positive populations.

FIG. 1B illustrates FACS analyses that using the same antibodies as in FIG. 1A, the presence of a buffer composition as provided herein reduces the prevalence of false double positive populations.

FIG. 2 illustrates FACS analyses using the same antibodies as in FIG. 1A, the presence of a buffer composition at higher concentrations of components reduces the prevalence of false double positive populations but may leave unacceptably high background.

FIG. 3, illustrates FACS analyses using the same antibodies as in FIG. 1A, the presence of a buffer composition at lower concentrations of components leaves the prevalence of false double positive populations.

FIG. 4 illustrates FACS analyses using buffers with differing types of polyethyelene glycol.

FIG. 5 illustrates FACS analyses using buffers at different concentrations relative to FIG. 4 and with differing types of polyethyelene glycol (PEG8K top row; PEG550 bottom row).

### DETAILED DESCRIPTION

Among the challenges remaining in florescent detection technologies, particularly in complex systems that employ polymer dyes, is the ability to combine multiple dyes in a single chamber in a way that provide stable interactions, but also prevents unwanted interactions with other antibodies or dye molecules in a system. One artifact of stacking is a reduction in overall signal due to improper placement of some antibodies in the detection system creating high background and often the presence of unwanted false double positive results. Disclosed are unique solution systems that enable not only specific binding of a target molecule, but also prevent dye stacking in complex antibody/dye cocktails thereby dramatically reducing observed background relative to systems without the provided buffer composition. Thus, the disclosed compositions and methods improve detectability of target analytes in complex systems.

The term “complex” as used herein is intended to mean a single solution that includes two or more antibody types wherein at least two different antibody types include differing dyes conjugated thereto.

When used to describe certain carbon atom-containing chemical groups, a parenthetical expression having the form “Cx-Cy” means that the unsubstituted form of the chemical group has from x carbon atoms to y carbon atoms, inclusive of x and y. For example, a C2-C4 alkyl is an alkyl group or radical having from 2 to 4 carbon atoms in its unsubstituted form. As another example, C1-C4 alkyl is an alkyl group or radical having from 1 to 4 carbon atoms in its unsubstituted form. A carbon atom-containing chemical group may be or include an alkyl, alkenyl, or alkynyl radical, and may be linear, branched or cyclic. Such carbon atom-containing chemical groups may be substituted or unsubstituted, and/or may include one or more substituent molecules in place of a hydrogen.

The term “alkyl” means a saturated straight or branched saturated hydrocarbon radical of from x to y carbon atoms. The carbons of the radical may be substituted at one or more hydrogens with a non-hydrogen molecule or group or unsubstituted.

The term “alkenyl” means an unsaturated straight or branched hydrocarbon radical of from x to y carbon atoms and includes at least one carbon-carbon double bond. The carbons of the radical may be substituted at one or more hydrogens with a non-hydrogen molecule or group or unsubstituted.

The terms “alkynyl” means an unsaturated straight or branched hydrocarbon radical of from x to y carbon atoms and includes at least one carbon-carbon triple bond. The carbons of the radical may be substituted or unsubstituted.

The term “cycloalkyl” means a saturated cyclic hydrocarbon radical of from x to y carbon atoms. For example, Cx-Cy cycloalkyl is defined as having from x to y carbon atoms. Examples of unsubstituted (C₃-C₈) cycloalkyl are cyclopropyl, cyclobutyl, cyclopentyl, cyclohexyl, cycloheptyl, cyclooctyl. A cyclic hydrocarbon radical may be or include an alkyl, alkenyl, or alkynyl radical. Such carbon atom-containing chemical groups may be substituted or unsubstituted, and/or may include one or more substituent molecules in place of a hydrogen.

The term “saturated” means lacking carbon-carbon double bonds, carbon-carbon triple bonds, and (in heteroatom-containing groups) carbon-nitrogen, carbon-phosphorous, carbo-oxygen or carbon-sulfur double bonds. The term “unsaturated” means containing one or more carbon-carbon double bonds, carbon-carbon triple bonds, and (in heteroatom-containing groups) carbon-nitrogen, carbon-phosphorous, carbon-oxygen, or carbon-sulfur double bonds.

The term “heteroatom,” refers to an atom other than hydrogen or carbon. Examples of groups containing one or more than one heteroatom include P(R^{P})₂, P(R^{P}), N(R^{N})₂, N(R^{N}), N, O, OR^{C}, S, SR^{C}, S(O), and S(O)₂, or other groups containing one or more of the foregoing wherein R is a linear, branched, or cyclic alkyl, alkenyl, or alkynl.

The term “heterocycle” means a cyclic, optionally fused cyclic, radical containing any number equal to or greater than 3, optionally 4-10, total ring atoms of which at least one atom is a carbon atom. The heteroatoms of the heterocycles may include N(R^{N})₂, N(R^{N}), N, O, OR^{C}, S, SR^{C}, S(O), and S(O)₂, wherein each of the heteroalkyl are unsubstituted or substituted and wherein R is a linear, branched, or cyclic alkyl, alkenyl, or alkynl. Examples of heterocycles with 4-6 total atoms (as an example) are oxetane, tetrahydofuran, morpholine, furan, piperazine, pyridine, thiazole, oxazole, tetrahydropyran, and thiomorpholine dioxide.

The term “substituted” means that at least one hydrogen atom (—H) bonded to a carbon atom or heteroatom of a corresponding unsubstituted compound or functional group is replaced by a substituent. Examples of such substituents are F, Cl, Br, OH, CF₃, NH₂, NHR^{N}, N(R^{N})₂. The term “—H” means a hydrogen or hydrogen radical that is covalently bonded to another atom. “Hydrogen” and “—H” are interchangeable, and unless clearly specified have identical meanings. R is a linear, branched, or cyclic alkyl, alkenyl, or alkynl.

The compositions disclosed herein have utility, e.g., as or improving studies using florescent detection molecules. As disclosed herein, a buffering composition includes one or more buffering agents, one or more celluloses or derivatives thereof, where the celluloses or derivatives thereof are at a concentration of less than about 2 wt % of the buffering composition, and a polyethylene glycol at less than about 2 wt % of the buffering composition. It was found that when multiple dyes, such as polymer dyes, are used on detection agents, that dye stacking led to unwanted stacking of the polymer dye molecules against each other when mixed and stored together, creating unacceptably high background and often some level of false double positive populations during analysis in fluorescence detection studies. The compositions as provided herein prevent such dye stacking and improve detectability of a target analyte.

A composition as provided herein is a buffering composition meaning that the composition includes at least one buffering agent that stabilizes pH of the system and resists changes in pH with acid or alkali is added to the buffering agent relative to a system without the buffering agent included. Choice of buffering agent can be readily handled by one of ordinary skill in the art, but buffering agents that have a pKa in the range of 6-8 are desirable for studies of many biological systems. Illustrative examples of such buffering agents include but are not limited to sodium bicarbonate, sodium carbonate, potassium bicarbonate, potassium carbonate, potassium phosphate, sodium phosphate, ethylene diamine, imidazole, triethanolamine, TRIS (tris(hydroxymethyl) aminomethane), HEPES(4-(2-Hydroxyethyl)-1-piperazineethanesulfonic acid), or any combination thereof. Optionally, a buffering agent excludes HEPES. A buffering agent is optionally present in the composition at a concentration of 0.01 millimolar (mM) to 10 molar (M), or any value or range therebetween. Optionally, a buffering agent is a phosphate buffering agent, illustratively sodium and/or potassium phosphates or mixture thereof as understood in the art optionally at 10 to 100 mM. Optionally, a buffering agent is phosphate buffered saline as this is understood in the art (e.g. 137 millimolar (mM) NaCl, 2.7 mM KCl, 10 mM Na2HPO4, and 1.8 mM KH2PO4 in water, or substantial equivalents thereof).

It was found that dye stacking could be reduced or eliminated by adding particular compounds to a buffering composition and improved to a greater extent when the particular compounds were at particular concentrations or ranges thereof. As an example, it was found that with the addition of one or more celluloses or derivatives thereof in combination with polyethylene glycol (PEG) wherein each of the celluloses or the PEG are present at no greater than 2 weight percent (wt %) of the buffering composition, that dye stacking such as that observed most particularly with polymer dyes is reduced or eliminated thereby dramatically reducing background.

As such, a buffering composition as provided herein includes one or more celluloses or derivatives thereof. Optionally, a buffering composition includes 1, 2, 3, 4 or more celluloses or derivatives thereof. Optionally, a buffering composition includes 1-3 celluloses or derivatives thereof. Optionally, a buffering composition includes 1-2 celluloses or derivatives thereof. Optionally, a buffering composition includes 1 cellulose or derivative thereof.

A cellulose is optionally a linear or branched alkyl cellulose. An alkyl cellulose optionally includes from hundreds to thousands of β(1→4) linked D-glucose units wherein one or more of the glucose units are modified by the addition of an alkyl on one or more hydroxyls in the structure. Optionally, an alkyl cellulose is a linear alkyl cellulose meaning that the alkyl cellulose includes one or more linear alkyl groups optionally excludes a branched alkyl. Optionally, an alkyl cellulose is a branched alkyl cellulose meaning that the alkyl cellulose includes one or more branched alkyl groups (e.g. isobutane). Optionally, an alkyl is bound to the glucose at a carbon on the alkyl that is not a terminal carbon. Optionally, a cellulose is a cellulose derivative. A cellulose derivative is optionally substituted at one or more positions with an alkyl, alkenyl, or alkynyl as defined herein, optionally a hydroxyalkyl, a carboxyalkyl, or other such derivative.

An alkyl on an alkyl cellulose is optionally a C₁-C₄ alkyl, optionally a C₁ alkyl, optionally a C₂ alkyl, optionally linear or branched a C₃ alkyl, optionally, a linear or branched C₄ alkyl. In some aspects, a cellulose is a methyl cellulose (MC) wherein one or more hydroxyls on the glucose is substituted with a C₁ alkyl. A methyl cellulose is optionally methyl cellulose with 12-18 cP measured at 2% in H₂O at 20° C. Optionally, a methyl cellulose is available from Sigma-Aldrich (St. Louis, MO) as product number M7027.

In other aspects, a cellulose is a hydroxypropyl cellulose (HC) wherein one or more of the hydroxyls on the glucose units are substituted with a hydroxylated C₃ alkyl. Optionally, a hydroxypropyl cellulose includes one or more CH₂CH₂(OH)CH₃ on 1, 2 or 3 hydroxyl locations on the glucose unit. Optionally, hydroxypropyl cellulose includes a CH₂CH₂(OH)CH₃ on three of the hydroxyl locations on the glucose unit. Optionally, a hydroxypropyl cellulose is of CAS number 9004-64-2, and optionally has a viscosity of 3-6 mPas, 2% in water at 20° C. and is optionally sold as product number H047325G by Fisher Scientific, Waltham, MA.

A cellulose is present in a composition at an amount of greater than zero to about 3 wt %. Optionally, a cellulose is present in a composition at from about 0.1 wt % to about 3.0 wt %. Optionally, a cellulose is present in a composition at from about 0.25 wt % to about 3.0 wt %. Optionally, a cellulose is present in a composition at from about 0.1 wt %, optionally about 0.2 wt %, optionally about 0.25 wt %, optionally about 0.3 wt %, optionally about 0.35 wt %, optionally about 0.4 wt %, optionally about 0.45 wt %, optionally about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %.

Optionally, a cellulose includes a methyl cellulose, the methyl cellulose present at from about 0.1 wt % to about 3.0 wt %. Optionally, a methyl cellulose is present in a composition at from about 0.25 wt % to about 3.0 wt %. Optionally, a methyl cellulose is present in a composition at from about 0.25 wt % to about 3 wt %. Optionally, a methyl cellulose is present in a composition at from about 2.0 wt % to about 3 wt %. Optionally, a methyl cellulose is present in a composition at from about 0.1 wt %, optionally about 0.2 wt %, optionally about 0.25 wt %, optionally about 0.3 wt %, optionally about 0.35 wt %, optionally about 0.4 wt %, optionally about 0.45 wt %, optionally about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %.

Optionally, a cellulose includes a hydroxypropyl cellulose, the hydroxypropyl cellulose present at from about 0.1 wt % to about 1.25 wt %. Optionally, a hydroxypropyl cellulose is present in a composition at from about 0.25 wt % to about 1 wt %. Optionally, a hydroxypropyl cellulose is present in a composition at from about 0.25 wt % to about 3.0 wt %. Optionally, a hydroxypropyl cellulose is present in a composition at from about 2.0 wt % to about 3.0 wt %. Optionally, a hydroxypropyl cellulose is present in a composition at from about 0.1 wt %, optionally about 0.2 wt %, optionally about 0.25 wt %, optionally about 0.3 wt %, optionally about 0.35 wt %, optionally about 0.4 wt %, optionally about 0.45 wt %, optionally about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %.

In some aspects, both a methyl cellulose and a hydroxypropyl cellulose are present in a buffering composition. Optionally, a methyl cellulose and a hydroxycellulose are each independently present in a composition at from about 0.1 wt % to about 1.25 wt %. Optionally, a methyl cellulose and a hydroxypropyl cellulose are each independently present in a composition at from about 0.25 wt % to about 1 wt %. Optionally, a methyl cellulose and a hydroxypropyl cellulose are each independently present in a composition at from about 2.0 wt % to about 3.0 wt %. Optionally, a methyl cellulose and a hydroxypropyl cellulose are each independently present in a composition at from about 0.1 wt %, optionally about 0.2 wt %, optionally about 0.25 wt %, optionally about 0.3 wt %, optionally about 0.35 wt %, optionally about 0.4 wt %, optionally about 0.45 wt %, optionally about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt % . . .

A buffering composition as provided herein optionally further includes one or more polymers of ethylene such as an ethylene glycol. The polyethylene glycol may be of any molecular weight, and may be branched or unbranched. An average molecular weight is optionally from about 10 kDa and about 100 kDa (the term “about” indicating that in preparations of polymer referred to herein with respect to polyethylene glycol, some molecules will weigh more, some less, than the stated molecular weight). As is typical in the polymer art, the provided molecular weight is an average molecular weight of the polymer material. In some aspects, a polyethylene glycol may be derivitzed meaning that the PEG may include one or more substituents on the polymer chain. Such substituents may be a C1-C4 alkyl, C1-C4 alkenyl, C1-C4 alkynl, carboxylic acid, phosphoric acid, or sulfonic acid derivatives, or any combination thereof. Suitable additional polymers are outlined in U.S. Pat. Nos. 5,795,569; 5,766,581; EP 01064951; U.S. Pat. No. 6,340,742; WO 00176640; WO 002017; EP0822199A2; WO 0249673A2; U.S. Pat. Nos. 4,002,531; 5,183,550; 5,985,263; 5,990,237; 6,461,802; 6,495,659; 6,448,369; 6,437,025; 5,900,461; 6,413,507; 5,446,090; 5,672,662; 6,214,966; 6,258,351; 5,932,462; 5,919,455; 6,113,906; 5,985,236; WO 9428024A1; U.S. Pat. Nos. 6,340,742; 6,420,339; and WO 0187925A2.

Optionally, a PEG is of a molecular weight exclusive of any substituent on the ethylene glycol backbone of about 100 Da, optionally about 1000 Da, optionally about 1500 Da, optionally about 2000 Da, optionally about 2500 Da, optionally about 3000 Da, optionally about 3500 Da, optionally about 4000 Da, optionally about 4500 Da, optionally about 5000 Da, optionally about 5500 Da, optionally about 6000 Da, optionally about 6500 Da, optionally about 7000 Da, optionally about 7500 Da, optionally about 8000 Da, optionally about 8500 Da, optionally about 9000 Da, optionally about 9500 Da, optionally about 10000 Da, optionally about 12000 Da, optionally about 15000 Da, optionally about 20000 Da. In some examples, a PEG is a polyethylene glycol monomethylether, optionally polyethylene glycol monomethylether 550. Optionally, a PEG is poly(ethylene glycol) 8000.

PEG is optionally present in a composition at a weight percent of greater than zero wt % to 3 wt % of said buffering composition. Optionally, PEG is present in a composition at 0.125 wt % to about 3 wt %, optionally 0.0.5 wt % to about 3 wt %, optionally 1.25 wt % to about 3 wt %. Optionally, PEG is present in a buffering composition at 0.1 wt %, optionally about 0.5 wt %, optionally about 0.2 wt %, optionally about 0.25 wt %, optionally about 0.3 wt %, optionally about 0.35 wt %, optionally about 0.4 wt %, optionally about 0.45 wt %, optionally about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %.

A buffering composition as provided herein optionally includes a combination of methyl cellulose and PEG. Optionally, composition includes about 0.1 wt % to 3.0 wt % methyl cellulose and about 0.1 wt % to about 3.0 wt % PEG. Optionally, a composition includes about 0.125 wt % to about 0.5 wt % PEG and methyl cellulose at about 0.25 wt %, optionally about 0.3 wt %, optionally about 0.4 wt %, optionally about 0.5 wt % optionally about 0.6 wt %, optionally about 0.7 wt %, optionally about 0.8 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %. In some aspects, a composition includes about 2.0 wt % to about 3.0 wt % methyl cellulose and about 0.125 wt % to about 3.0 wt % PEG, optionally about 2.5 wt % methyl cellulose and about 1.25 wt % PEG.

A buffering composition as provided herein optionally includes a combination of hydroxypropyl cellulose and PEG. Optionally, composition includes about 0.1 wt % to 3.0 wt % hydroxypropyl cellulose and about 0.1 wt % to about 1.0 wt % PEG. Optionally, a composition includes about 0.125 wt % to about 3.0 wt % PEG and hydroxypropyl cellulose at about 0.25 wt %, optionally about 0.3 wt %, optionally about 0.4 wt %, optionally about 0.5 wt % optionally about 0.6 wt %, optionally about 0.7 wt %, optionally about 0.8 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %. In some aspects, a composition includes about 2.0 wt % to about 3.0 wt % hydroxypropyl cellulose and about 0.125 wt % to about 3.0 wt % PEG, optionally about 2.5 wt % hydroxypropyl cellulose and about 1.25 wt % PEG.

A buffering composition as provided herein optionally includes a combination of methyl cellulose, hyrdoxypropyl cellulose and PEG. Optionally, composition includes about 0.1 wt % to 3.0 wt % methyl cellulose, about 0.1 wt % to about 3.0 wt % hydroxypropyl cellulose and about 0.1 wt % to about 3.0 wt % PEG. Optionally, a composition includes about 0.125 wt % to about 3.0 wt % PEG, methyl cellulose at about 0.25 wt %, optionally about 0.3 wt %, optionally about 0.4 wt %, optionally about 0.5 wt % optionally about 0.6 wt %, optionally about 0.7 wt %, optionally about 0.8 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %, and hydroxypropyl cellulose at about 0.25 wt %, optionally about 0.3 wt %, optionally about 0.4 wt %, optionally about 0.5 wt % optionally about 0.6 wt %, optionally about 0.7 wt %, optionally about 0.8 wt %, optionally about 0.9 wt %, optionally about 1.0 wt %, optionally about 1.1 wt %, optionally about 1.2 wt %, optionally about 1.25 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %, optionally about 2.1 wt %, optionally about 2.2 wt %, optionally about 2.3 wt %, optionally about 2.4 wt %, optionally about 2.5 wt %, optionally about 2.6 wt %, optionally about 2.7 wt %, optionally about 2.8 wt %, optionally about 2.9 wt %, optionally about 3.0 wt %. Optionally, a composition includes about 2.5 wt % methyl cellulose, about 2.5 wt % hydroxypropyl cellulose, and about 1.25 wt % PEG. In some aspects, a composition includes about 0.5 wt % methyl cellulose, about 0.5 wt % hydroxypropyl cellulose, and about 0.125 wt % to about 0.5 wt % PEG. Optionally, the composition includes about 0.25 wt % to 1.0 wt % methyl cellulose and about 0.25 wt % to 1.0 wt % hydroxypropyl cellulose, and about 0.125 wt % to about 0.5 wt % polyethylene glycol.

In some aspects, a buffering composition includes an alcohol, optionally a polyvinyl alcohol. Optionally, a composition excludes an alcohol, optionally a polyvinyl alcohol. In some aspects, an alcohol is a polyvinyl alcohol (PVA). Polyvinyl alcohol is a homopolymer of ethanol and includes a predominant of 1,3-diol linkages but may also include some level of 1,2-diol linkages. In some aspects, PVA is CAS 9002-89-5 (44.05 g/mol; Fisher Scientific, Waltham MA). PVA is optionally present in a composition at a weight percent of greater than zero wt % to 2 wt % of said buffering composition. Optionally, PVA is present in a buffering composition at 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %.

It is appreciated that a buffering composition optionally further includes one or more salts as are typically included in a buffer as understood in the art, optionally NaCl, KCl, MgCl₂, among others, or combinations thereof. A salt is optionally present at a concentration of 0.1 mM to 250 mM, or any value or range therebetween.

Not all compounds are suitable for use in a buffering composition as provided herein. For example, certain salts such as CaCl₂) or ammonium chloride negatively affect the ability to reduce or eliminate stacking or otherwise negatively effects assay performance. Some other compounds have no effect or a negative on reducing or eliminating stacking or production of double positive populations of cells due to stacking interactions between dye molecules. For example, a buffer composition optionally excludes CaCl₂), ammonium chloride, diazolidinyl urea, dinitrobenzenesulfonate (DNBS), bis-hydroxy urea, formaldehyde, propyl gallate, saponin, bishydroxy methyl urea, casein, a dextran, polyethylene glycol, glucose (free glucose), 4-((4-(dimethylamino)phenyl) azo)benzoyl(Dabcyl), oligonucleotides (e.g. QSY® 21, ssDNA), HEPES (some aspects), tween, ethanol, heparin, phloretin, polyvinylpyrrolidone (PVP), ethylenediaminetetraacetic acid (EDTA), sulfuric acid, carrageenan, CHAPS((3-((3-cholamidopropyl)dimethylammonio)-1-propanesulfonate), dimethyl sulfoxide (DMSO), dimethyl formamide, fetal bovine serum, or any combination thereof.

In some aspects, a buffering composition further includes one or more additives. An additive is optionally a sugar, a protein additive, or the like, or combinations thereof. Illustrative examples of an additive include a sugar, optionally fructose, sucrose, or the like. When present, a sugar may be included at about 0.1 wt % to about 5 wt % or other desirable concentration. In some aspects, a sugar is present at about 0.1 wt %, optionally about 0.25 wt %, optionally about 0.5 wt %, optionally about 0.75 wt %, optionally about 1.0 wt %, optionally about 1.25 wt %, optionally about 1.5 wt %, optionally about 1.75 wt %, optionally about 2.0 wt %, optionally about 2.25 wt %, optionally about 2.5 wt %, optionally about 2.75 wt %, optionally about 3.0 wt %, optionally about 3.25 wt %, optionally about 3.5 wt %, optionally about 3.75 wt %, optionally about 4.0 wt %, optionally about 4.25 wt %, optionally about 4.5 wt %, optionally about 4.75 wt %, optionally about 5.0 wt %.

An additive is optionally a protein additive. Illustrative examples of a protein additive include but are not limited to an albumin or the like. A protein additive is optionally an albumin, optionally bovine serum albumin or human serum albumin. A protein additive is optionally included at about 0.1 wt % to about 5 wt % or other desirable concentration. In some aspects, a protein additive is present at about 0.1 wt %, optionally about 0.25 wt %, optionally about 0.5 wt %, optionally about 0.75 wt %, optionally about 1.0 wt %, optionally about 1.25 wt %, optionally about 1.5 wt %, optionally about 1.75 wt %, optionally about 2.0 wt %, optionally about 2.25 wt %, optionally about 2.5 wt %, optionally about 2.75 wt %, optionally about 3.0 wt %, optionally about 3.25 wt %, optionally about 3.5 wt %, optionally about 3.75 wt %, optionally about 4.0 wt %, optionally about 4.25 wt %, optionally about 4.5 wt %, optionally about 4.75 wt %, optionally about 5.0 wt %.

The remainder of the buffering composition optionally includes water, optionally purified water or any water suitable for use in biological analyses such as flow cytometry and the like.

A buffering composition as used herein may be combined with one or more analytical agents optionally for the detection, characterization, purification, or observation of a target analyte in a system. An analytical agent is optionally any molecule that may be used to bind to a target, optionally selectively bind a target optionally wherein the K_{d} for the desired target is at least 10-fold lower for the target than for any other molecule naturally found in a organism. A target is any structure desired to be labeled, optionally a tissue, a cell, a protein, a nucleic acid, a lipid, or any other desired structure.

An analytical agent is any molecule that may bind a target, and optionally, selectively bind a target. An analytical agent may be a protein, nucleic acid (double or single stranded DNA, RNA, or other), hormone, neurotransmitter, sugar, organic or inorganic small molecule (e.g. 1000 Da or smaller), or other material that is able to selectively bind a target. A polypeptide is optionally any molecule with 50 or fewer amino acids in a continuous chain. Illustrative examples of an analytical agent. Illustrative examples of an analytical agent include, but are not limited to an antibody, such as any suitable antibody known in the art including other immunologically active fragments of antibodies or single chain antibodies. An analytical agent may be a single moiety, e.g., a polypeptide or protein, or it may include two or more moieties, e.g., a pair of polypeptides such as a pair of single chain antibody domains. An antibody may be a monoclonal antibody or a polyclonal antibody. An antibody or fragment thereof may be derived from an organism such as sheep, goat, rabbit or rat.

An analytical agent may be an antibody fragment such as single chain antibodies (scFv), Fab and scFv antibodies, single domain antibodies (VHH), or chimeric antibodies. The analytical agent may be derived from a naturally occurring protein or polypeptide; it may be designed de novo, or it may be selected from a library. Optionally, an analytical agent may be derived from an antibody, a single chain antibody (scFv), a single domain antibody (VHH), a lipocalin, a single chain MHC molecule, an Anticalin™ (Pieris), an Affibody™, a nanobody (Ablynx) or a Trinectin™ (Phylos).

In some aspects, an analytical agent is a fragment of an antibody, optionally an antigen binding fragment or a variable region. Examples of antibody fragments include Fab, Fab′, F(ab′)₂ and Fv fragments. Additional analytical agent molecules include diabodies, linear antibodies, single-chain antibody molecules, and multispecific antibodies formed from antibody fragments.

An analytical agent may be linked to one or more dyes. A dye as used herein may be a fluorophore. A fluorophore may be a rhodamine dye, a fluorescein dye, a coumarin dye, an indole dye, or any combination thereof. Optionally, a dye may be or include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.

It was found that the buffering composition as used herein is particularly suited to the use of polymer dyes, many of which are themselves often subject to stacking interactions, which are reduced or eliminated by the use of the presence buffering composition. Illustrative examples of polymer dyes that may be used in any aspect provided herein include but are not limited to those described in U.S. Pat. No. 8,354,239, International application publication numbers WO2004/000970 or WO 2020/187919, or dyes sold as Brilliant Violet™ dyes by BD Biosciences (Franklin Lakes, NJ). In some aspects, the dye is Brilliant Violet 421™, Brilliant Violet 570™ Brilliant Violet 605™, Brilliant Violet 650™, Brilliant Violet 711™, or Brilliant Violet 785™.

As such, also provided are cocktails that can be used for analyzing a biological sample wherein the cocktail includes a buffer composition as provided herein combined with one or more analytical agents conjugated to one or more dyes. It was found that the buffer composition as provided herein is capable of reducing or eliminating dye stacking interactions thereby improving analytical results. The use of a buffering composition as provided herein prevents dye stacking interactions that occur in the absence of the buffer composition as provided herein such that when a cocktail is combined with a biological sample, the analytical agent is more effectively able to specifically bind a target and dramatically reduce or eliminate background and in some aspects, also false double positive populations that are produced by stacking of multiple target analytes.

A cocktail as provided herein includes a buffer composition as provided herein and one or more analytical agents bound to a dye molecule, and optionally one or more additives as provided herein. Optionally, an analytical agent is an antibody, fragment hereof, or derivative thereof, as provided herein and conjugated to one or more dye molecules, optionally polymer dyes.

Also disclosed herein are methods of detecting the presence or absence of an analyte/target in a sample (optionally a biological sample) or an organism. The methods may include contacting a sample with a buffer composition or cocktail as disclosed herein, to form an analysis composition and subjecting the analysis composition to analysis by detecting one or more of the dyes in contact with a portion of the biological sample. The detecting may utilize one or more various analytical methods illustratively but not limited to flow cytometry, FISH, immunohistochemistry, sandwich assay, Southern blot, western blot, microarray, or substrate binding assay.

Optionally, a sample is a biological sample containing one or a plurality of cells. Such a biological sample is optionally a cell or tissue or plurality thereof. In some aspects, a biological sample is plasma, serum, whole blood, saliva, urine, feces, lymph, nasal secretions, or other desired sample. In some aspects, a biological sample is whole blood or plasma. Optionally, a sample is plasma. An analysis composition may be formed by contacting a biological sample to a cocktail as provided herein thereby contacting the analytical agent in the cocktail with the target within the biological sample. In other aspects, the cocktail is contacted with a biological agent forming an analysis composition. Following contacting of the biological sample with the cocktail, the analysis composition is subjected to analysis by detecting one or more analytical agents in contact with a portion of said biological sample.

In some aspects, a method includes forming an analysis composition by contacting a biological sample comprising plasma or whole blood with a cocktail comprising the buffer composition as provided herein and one or more antibodies, said one or more antibodies conjugated to one or more florescent dyes, optionally polymer dyes, and subjecting the analysis composition to analysis by flow cytometry.

In some aspects, the cocktail used in a method of analyzing a biological sample includes two or more antibodies or derivatives thereof specific for different target molecules, wherein an antibody or derivative thereof specific for a first target molecule is conjugated to a first dye, and wherein an antibody or derivative thereof specific for a second target molecule is conjugated to a second dye, where the first dye and the second dye are characterized by different fluorescent properties (e.g. absorption wavelength width, emission wavelength width, absorption maximum, emission maximum, Stokes shift, lifetime, anisotropy, photon yield, signal intensity, etc.). Optionally, a first dye, a second dye or both are a polymer dye. Optionally, both a first dye and a second dye are polymer dyes. Optionally, a cocktail includes 2-23 different antibodies or derivatives thereof each conjugated with a different dye, optionally including at least one polymer dye, optionally a brilliant violet dye. In some aspects, at least one antibody is conjugated with a polymer dye (optionally Brilliant Violet 421™, Brilliant Violet 570™, Brilliant Violet 605™, Brilliant Violet 650™, Brilliant Violet 711™, or Brilliant Violet 785™), and at least one antibody is conjugated with a different dye type, optionally a rhodamine dye, a fluorescein dye, a coumarin dye, an indole dye, or any combination thereof.

Various aspects of the present disclosure are illustrated by the following non-limiting examples. The examples are for illustrative purposes and are not a limitation on any practice of the present disclosure. It will be understood that variations and modifications can be made without departing from the spirit and scope of the disclosure. Reagents illustrated herein are commercially available or readily synthesized by well-known methods from readily commercially available precursors, and a person of ordinary skill in the art readily understands where such reagents may be obtained.

### EXAMPLES

### Example 1

Buffering compositions were made using various amounts of methyl cellulose (M7027; Millipore), hydroxypropyl cellulose (H047325G; Fisher Scientific, Waltham, MA), or both and combined with either polyethylene glycol monomethylether 550 (PEG550) CAS No: 9004-74-4 with molecular weight 525-575 Da available from ThermoFisher or poly(ethylene glycol) 8000 (PEG8K) CAS No: 107-21-1 with hydroxyl value of 12 to 16 mg KOH/g available from ThermoFisher. in water. 5 wt % stock solutions were prepared for each of methyl cellulose and hydroxypropyl cellulose by adding 2.5 grams (g) to 1X PBS((0.87 wt % sodium chloride, 0.14 wt % Sodium phosphate monobasic), respectively. 2.5 g of each compound was partially dissolved in 35 ml PBS in a 50 ml conical tube and placed on a rocker for 3-4 hours. Then the tubes were transferred into a refrigerator overnight. The next day, volumes were adjusted to 50 ml with 1X PBS. A first buffer composition was made by combining the stock solutions to form three test solutions: 1) 3 wt % methyl cellulose, 3 wt % hydroxypropyl cellulose and 3 wt % PEG; 2) 0.5 wt % methyl cellulose, 0.5 wt % hydroxypropyl cellulose and 0.25 wt % PEG; and 3) 0.03 wt % methyl cellulose, 0.03 wt % hydroxypropyl cellulose and 0.03 wt % PEG. Separate solutions were made with polyethylene glycol monomethylether 550 or poly(ethylene glycol) 8000, with the MC and HC at all the forgoing concentrations. A second buffer composition was made by combining the stock solutions to form a 1 wt % hydroxypropyl cellulose and 0.5 wt % PVA solution.

Individual brilliant violet dye (BV) conjugated antibodies were marinated by incubating the antibody (0.1-0.5 μg) with each tested buffer composition by adding the antibody to the buffer composition in appropriate concentrations in a smallest volume possible. 0.5 wt % of Bovine Serum Albumin (BSA) in PBS(1X) was used to adjust the mixture to the final volume. Antibodies tested were anti-CD19 conjugated to Brilliant Violet™ 711 (BV711), anti-CD4 conjugated to Brilliant Violet™ 421 (BV421), anti-CD4 conjugated to Brilliant Violet™ 750 (BV750), anti-CD56 conjugated to Brilliant Violet™ 737 (BV737), anti-CD8 conjugated to Brilliant Violet™ 605 (BV605), anti-CD8 conjugated to Brilliant Violet™ 570 (BV570), anti-CD8 conjugated to Brilliant Violet™ 711 (BV711), anti-CD28 conjugated to Brilliant Violet™ 785 (BV785), anti-CD14 conjugated to Brilliant Violet™ 785 (BV785), anti-CD16 conjugated to Brilliant Violet™ 421 (BV421). Antibodies were obtained from Cytek Biosciences and conjugated by standard techniques.

Cocktails were formulated either using “marinated” BV-conjugated antibodies and “non-marinated” non-BV-conjugated antibodies or “non-marinated” BV and non-BV antibodies. In both cases, the final volume was 50 μl. Single test volumes were dispensed into FACS tubes, to the center of the bottom of the tube.

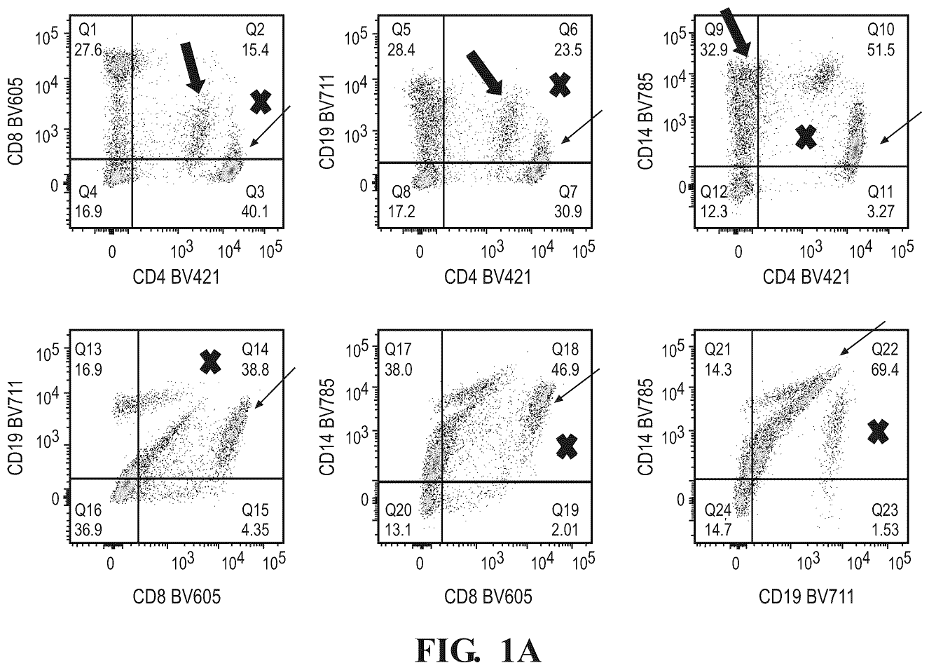

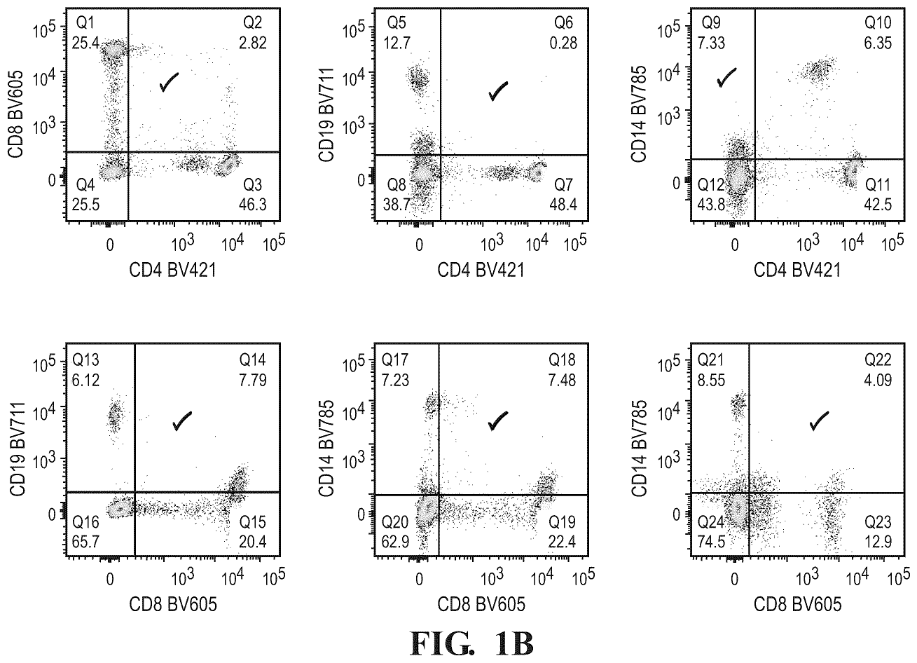

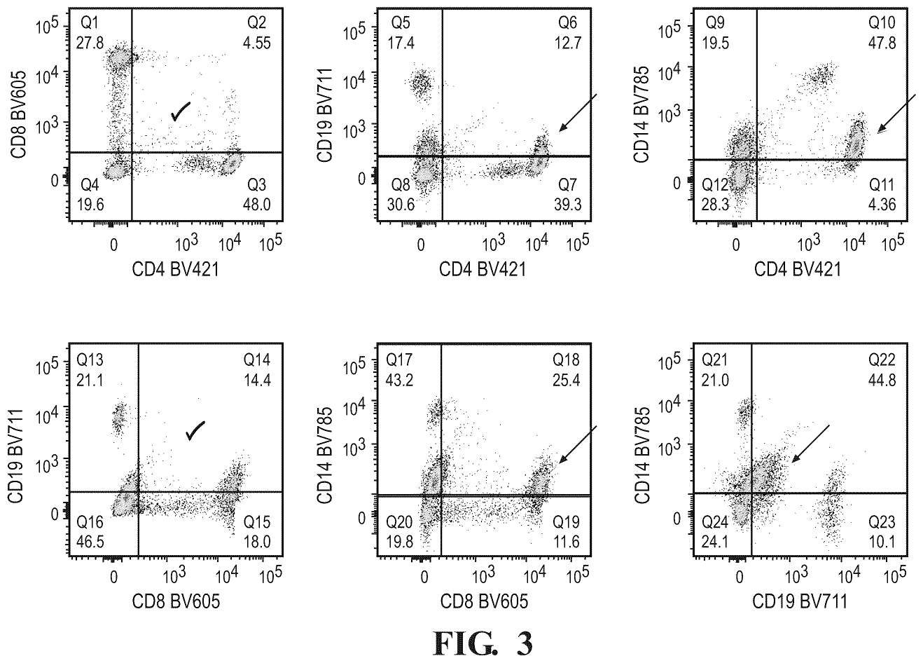

Human whole blood was obtained from consenting donors and used as a biological sample for studies of the ability of a buffer composition as provided herein to reduce or eliminate high background and in some cases the presence of false double populations using the above antibody cocktails. 10 μl of the respective buffer composition is added to each study tube followed by the addition of desired antibodies and bringing volume up to 50 μl using cell stain buffer. 100 μl whole blood was added to each tube and incubated in the dark at ambient temperature for 15 min. 2 ml of 1X lysis buffer was added and incubated in the dark at ambient temperature for 15 min. The tubes were spun at 1500×g for 5 min. The supernatant was decanted and the pellet washed 2× with wash buffer. To this about 300 μl fix was added and the sample run on the flow cytometer using single color beads for compensation. Results gating on desired populations are illustrated in FIGS. 1-5.

FIG. 1A illustrates results using dye-conjugated antibodies in standard PBS buffer. The flow cytometer output was gated on lymphocytes and monocytes. A narrow arrow illustrates false double positives and a thick arrow demonstrates high background. An X indicates unacceptable results. With each of the tested dye-conjugated antibodies, false double positive populations were observed and many antibody combinations showed unacceptably high background. When a buffer composition of 0.5 wt % methyl cellulose, 0.5 wt % hydroxypropyl cellulose, and 0.25 wt % PEG550 was added to the system prior to marinating with BV-conjugated antibodies, both the high background and the false double positive populations were no longer observed as illustrated in FIG. 1B with a check mark illustrating acceptable results.

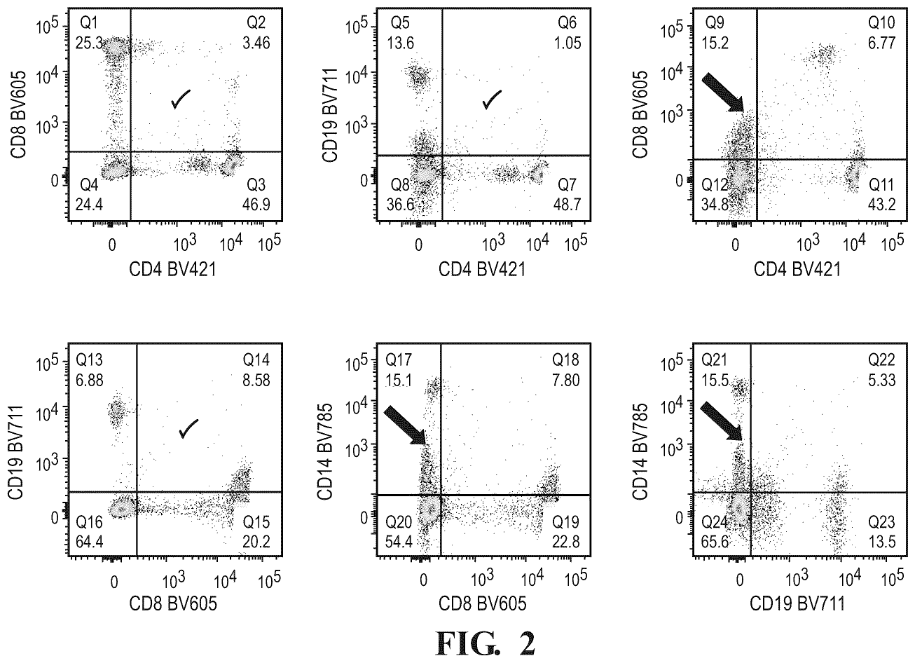

To test various concentrations of elements of a buffer composition, additional studies were preformed using a buffer composition including 3 wt % methyl cellulose, 3 wt % hydroxypropyl cellulose and 3 wt % PEG550. As is illustrated in FIG. 2, while some combinations of antibodies showed acceptable background and no false double positive populations, others showed unacceptably high background showing that the concentrations used were suboptimal.

Similarly, a low concentration buffer solution was also tested. The low concentration buffer composition included 0.3 wt % methyl cellulose, 0.3 wt % hydroxypropyl cellulose and 0.3 wt % PEG550. Once again, while a couple antibody combinations showed acceptable results, high levels of false double positive populations were also observed in several other antibody combinations illustrating that the concentrations of elements in the buffer composition were suboptimal.

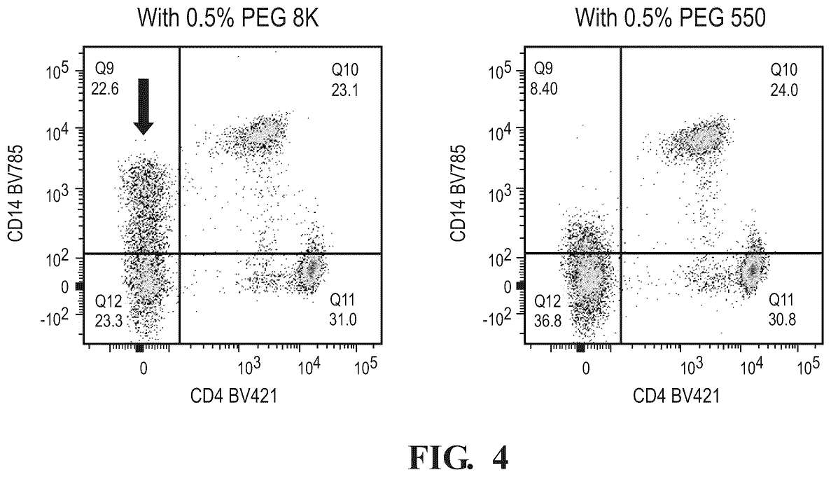

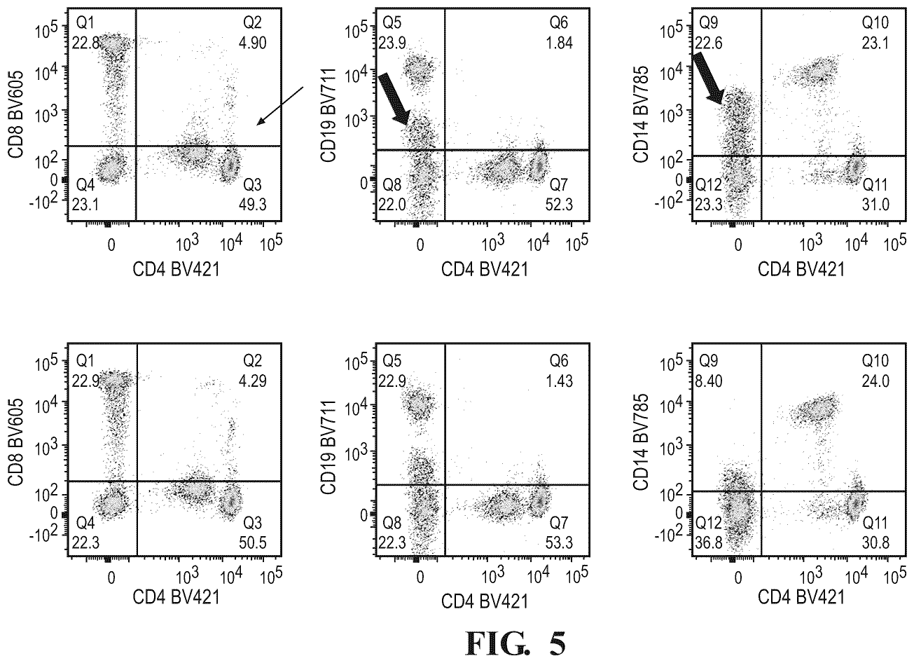

The buffer compositions were also formulated with 0.5 wt % methyl cellulose, 0.5 wt % hydroxypropyl cellulose and 0.25 wt % PEG8K and compared with the same formulation using PEG550. As is illustrated in FIG. 4, PEG8K left some level of high background that was eliminated using buffer with PEG550 showing that PEG550 was superior to PEG8K. Similar results are illustrated at 0.25 wt % methyl cellulose, 0.25 wt % hydroxypropyl cellulose and 0.5 wt % PEG8K (top row) or 0.5 wt % PEG550 (bottom row) as illustrated in FIG. 5.

Various other combinations of elements in a buffer composition were tested and shown not to be optimal yet could be at least partially rescued by the presence of polyethylene glycol. Results are illustrated in Table 1.

TABLE 1

| MC | HC | PVA | PEG550 | Result |
|---|---|---|---|---|
| X | | X | | high background and false double |
| | | | | positive populations |
| X | | X | X | reduced background, but remained false |
| | | | | double positive populations |
| | X | X | | high background and false double |
| | | | | positive populations |
| | X | X | X | reduced background, but remained false |
| | | | | double positive populations |
| X | X | | | high background and false double |
| | | | | positive populations |
| X | X | | X | reduced background and eliminated false |
| | | | | double positive populations. |

MC—methyl cellulose; HC—hydroxypropyl cellulose; PVA—polyvinyl alcohol; X indicates presence of reagent.

### Additional Exemplary Aspects

Aspect 1. A buffering composition including:
one or more buffering agents;
one or more celluloses or derivatives thereof, the celluloses or derivatives thereof at a concentration of less than about 3 wt % of the buffering composition; and
a polyethylene glycol, the polyethylene glycol present at less than or equal to about 3 wt % of said buffering composition.

Aspect 2. The buffering composition of Aspect 1, wherein the cellulose is a linear or branched alkyl cellulose.

Aspect 3. The buffering composition of any of Aspects 1 or 2, wherein the alkyl cellulose is a C₁-C₄ alkyl cellulose.

Aspect 4. The buffering composition of any of Aspect 2 or 3, wherein the alkyl cellulose is methyl cellulose.

Aspect 5. The buffering composition of Aspects 1-4, wherein the cellulose is a branched chain alkyl cellulose.

Aspect 6. The buffering composition of any of Aspects 1-5, wherein the cellulose is hydroxypropyl cellulose.

Aspect 7. The buffering composition of any of Aspect 1, wherein the cellulose is a combination of methyl cellulose and hydroxypropyl cellulose.

Aspect 8. The buffering composition of any of Aspects 1-7, wherein the cellulose is present at about 0.1 wt % to about 3.0 wt %.

Aspect 9. The buffering composition of any of Aspects 1-7, wherein the cellulose is present at about 0.25 wt % to about 3.0 wt %.

Aspect 10. The buffering composition of any of Aspects 1-9, wherein the buffering agent is characterized by a pKa of 6-8.

Aspect 11. The buffering composition of any of Aspects 1-10, wherein said buffering agent comprises a phosphate.

Aspect 12. The buffering composition of any of Aspects 1-11, wherein the buffering agent is phosphate buffered saline.

Aspect 13. The buffering composition of any of Aspects 1-12, wherein the composition further comprises an albumin, optionally bovine serum albumin or human serum albumin.

Aspect 14. The buffering composition of any of Aspects 1-13, wherein the composition includes about 0.1 wt % to 3 wt % methyl cellulose, about 0.01 wt % to 3 wt % hydroxypropyl cellulose, and about 0.1 wt % to about 3 wt % polyethylene glycol.

Aspect 15. The buffering composition of any of Aspects 1-13, wherein the composition includes about 0.25 wt % to 3 wt % methyl cellulose and about 0.25 wt % to 3 wt % hydroxypropyl cellulose.

Aspect 16. The buffering composition of any of Aspects 1-13, wherein the composition includes about 2.0 wt % to 3.0 wt % methyl cellulose and about 2.0 wt % to 3 wt % hydroxypropyl cellulose, and about 0.125 wt % to about 3 wt % polyethylene glycol.

Aspect 17. The buffering composition of claim 1, wherein said composition comprises about 2.5 wt % methyl cellulose and about 2.5 wt % hydroxypropyl cellulose, and further comprising about 0.125 wt % to about 3 wt % polyethylene glycol.

Aspect 18. The composition of any of Aspects 1-17, wherein the polyethylene glycol is polyethylene glycol monomethylether 550, poly(ethylene glycol) 8000, or a combination thereof.

Aspect 19. A method of analyzing a biological sample comprising:
obtaining a biological sample,
forming an analysis composition by contacting said biological sample with a cocktail comprising the buffer composition of any one of claims 1-18 and one or more antibodies, said one or more antibodies conjugated to one or more florescent dyes; and
subjecting said analysis composition to analysis by detecting one or more of said dyes in contact with a portion of said biological sample.

Aspect 20. The method of Aspect 19, wherein the cocktail includes two or more antibodies specific for different target molecules, wherein an antibody specific for a first target molecule is conjugated to a first dye, and wherein an antibody specific for a second target molecule is conjugated to a second dye, the first dye and said second dye characterized by different fluorescent properties.

Aspect 21. The method of any of Aspects 19-20, including 1 to 23 antibodies, optionally 2 to 23 antibodies, directed to different target molecules and each conjugated to a different dye.

Aspect 22. The method of any of Aspects 19-21, wherein at least two of the antibodies are conjugated to different brilliant violet dyes.

Aspect 23. The method of any of Aspects 19-22, wherein the dyes include one or more polymer dyes, optionally Brilliant Violet 421™, Brilliant Violet 510™, Brilliant Violet 570™, Brilliant Violet 605™, Brilliant Violet 650™, Brilliant Violet 711™, Brilliant™ Violet 750, or Brilliant Violet 785™.

Aspect 24. The method of any of Aspects 19-23, wherein one or more dyes include a rhodamine dye, a fluorescein dye, a coumarin dye, an indole dye, or any combination thereof.

Aspect 25. The method of Aspect 24, wherein said dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY™ FL-Br2, BODIPY™ 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.

Aspect 26. The method of any of Aspects 19-25, including drying the analysis sample, wherein the cocktail includes 0.1 wt % to 3 wt % methyl cellulose, about 0.01 wt % to 3 wt % hydroxypropyl cellulose, and about 0.1 wt % to about 3 wt % polyethylene glycol.

Aspect 27. The method of any of Aspects 19-25, wherein the cocktail includes about 0.25 wt % to 3 wt % methyl cellulose and about 0.25 wt % to 3 wt % hydroxypropyl cellulose, and about 0.125 wt % to about 3 wt % polyethylene glycol.

Aspect 28. The method of any of Aspects 19-25, wherein the cocktail includes about 2 wt % to about 3 wt % methyl cellulose and about 2 wt % to about 3 wt % hydroxypropyl cellulose, and further comprising about 0.125 wt % to about 3 wt % polyethylene glycol.

Aspect 29. The method of any of Aspects 19-28, wherein the subjecting is by flow cytometry, FISH, immunohistochemistry, sandwich assay, Southern blot, western blot, microarray, or substrate binding assay.

Aspect 30. The method of any of Aspects 19-28, wherein the subjecting is by flow cytometry.

Aspect 31. A method of forming an analysis composition for use in analysis of a biological sample including:
combining an analysis composition by contacting the biological sample with a cocktail including the composition of any one of Aspects 1-18 and one or more antibodies, the one or more antibodies conjugated to one or more florescent dyes.

Aspect 32. The method of Aspect 31, wherein the cocktail includes 0.1 wt % to 3 wt % methyl cellulose, about 0.01 wt % to 3 wt % hydroxypropyl cellulose, and about 0.1 wt % to about 3 wt % polyethylene glycol.

Aspect 33. The method of any of Aspects 31-32, wherein the dyes include one or more polymer dyes, optionally Brilliant Violet 421™, Brilliant Violet 570™, Brilliant Violet 605™, Brilliant Violet 650™, Brilliant Violet 711™, or Brilliant Violet 785™.

Aspect 34. The method of any of Aspects 31-32, wherein one or more dyes include a rhodamine dye, a fluorescein dye, a coumarin dye, an indole dye, or any combination thereof.

### SELECTED DEFINITIONS

2-[3-[3-[6-[(2,5-dioxo-1-pyrrolidinyl)oxy]-6-oxohexyl]-2 (3H)-benzoxazolylidene]-1-propen-1-yl]-3-ethyl-benzoxazolium, monoiodide (Cy2), Sulfo-Cyanine3 (Cy3), Cyanine 3.5 (Cy3.5), Sulfo-Cyanine5 (Cy5), Cyanine 5.5 (Cy5.5), Sulfo-Cyanine7 (Cy7), PerCP (peridinin chlorophyll-a Protein), JOE (6-carboxy-4′,5′-dichloro-2′,7′-dimethoxyfluorescein), ROX (5-(and -6)-carboxy-X-rhodamine), 7-amino-3-[2-(2,5-dioxopyrrolidin-1-yl)oxy-2-oxoethyl]-4-methyl-2-oxochromene-6-sulfonic acid (Alexa Fluor® 350), N,N-Diethylethanaminium-[9-{6-[(2,5-dioxo-1-pyrrolidinyl)oxy]-6-oxohexyl}-8,8-dimethyl-2-oxo-4-(trifluormethyl)-8,9-dihydro-2H-pyrano[3,2-g]chinolin-6-yl]methansulfonate (Alexa Fluor® 430), dilithium; 3-(3-amino-6-imino-5-sulfo-4-sulfonatoxanthen-9-yl)-4-carboxybenzoate (Alexa Fluor® 488), ihydrogen 5-(4-{[(2,5-dioxo-1-pyrrolidinyl)oxy]carbonyl}phenyl)-2,3,3,7,7,8-hexamethyl-2,3,7,8-tetrahydro-1H-pyrrolo[3′,2′: 6, 7]chromeno [3,2-f]indole-10,12-disulfonate (Alexa Fluor® 532), sodium 6-(2-carboxy-3,4,6-trichloro-5-{[2-({6-[(2,5-dioxopyrrolidin-1-yl)oxy]-6-oxohexyl}amino)-2-oxoethyl]thio}phenyl)-2,2,4,8,10,10-hexamethyl-3,4,5a,8,9,10,11,12a-octahydro-2H-pyrano[3,2-g: 5,6-g′]diquinolin-1-ium-12,14-disulfonate (Alexa Fluor® 546), 4-(2,5-dioxopyrrolidin-1-yl)oxycarbonyl-2-[7,7,19,19-tetramethyl-9,17-bis(sulfomethyl)-2-oxa-6,20-diazapentacyclo[12.8.0.03,12.05,10.016,21]docosa-1 (14),3,5,8,10,12,15,17,21-nonaen-yl]benzoic acid (Alexa Fluor® 568), [13-[2-carboxy-4-(2,5-dioxopyrrolidin-1-yl)oxycarbonylphenyl]-6,7,7,19,19,20-hexamethyl-17-(sulfomethyl)-2-oxa-20-aza-6-azoniapentacyclo[12.8.0.03,12.05,10.016,21]docosa-1 (14),3,5,8,10,12,15,17,21-nonaen-9-yl]methanesulfonate (Alexa Fluor® 594), Alexa Fluor® 633 found at CHEBI: 137393, 3-[(2Z)-2-[(2E,4E)-5-[3,3-dimethyl-5-sulfo-1-(3-sulfopropyl) indol-1-ium-2-yl]penta-2,4-dienylidene]-3-[5-(2,5-dioxopyrrolidin-1-yl)oxy-5-oxopentyl]-3-methyl-5-sulfoindol-1-yl]propane-1-sulfonate (Alexa Fluor® 647), 7-amino-4-methylcoumarin-3-acetic acid, (3-{2-[(3,5-Dimethyl-1H-pyrrol-2-yl-kappaN)methylene]-2H-pyrrol-5-yl-kappaN}propanoato) (difluoro) boron (BODIPY® FL), CAS No. 216961-93-2 (BODIPY® 530/550), 12-(2,2-difluoro-12-thiophen-2-yl-3-aza-1-azonia-2-boranuidatricyclo[7.3.0.03,7]dodeca-1 (12),4,6,8,10-pentaen-4-yl)dodecanoate; hydron (BODIPY® 558/568), CAS #: 150173-90-3 (BODIPY® 564/570), CAS #: 150173-78-7 (BODIPY® 576/589), CAS Number: 217075-36-0 (BODIPY® 581/591), CAS #: 2183512-02-7 (BODIPY® 630/650), CAS No.: 1818267-45-6 (BODIPY® 650/665), Difluoro (3-{2-[(5-phenyl-1H-pyrrol-2-yl-kappaN)methylene]-2H-pyrrol-5-yl-kappaN}propanoato) boron (BODIPY® R6G), CAS #: 485397-12-4 (BODIPY® TMR), CAS #: 2183473-18-7 (BODIPY® TR).

The foregoing description of particular aspect(s) is merely exemplary in nature and is in no way intended to limit the scope of the disclosure, or any related invention, its application, or uses, which may, of course, vary. The compositions or processes are described with relation to the non-limiting definitions and terminology included herein. These definitions and terminology are not designed to function as a limitation on the scope or practice of any invention disclosed herein but are presented for illustrative and descriptive purposes only. While the processes or compositions are described as an order of individual steps or using specific materials, it is appreciated that steps or materials may be interchangeable such that the description may include multiple parts or steps arranged in many ways as is readily appreciated by one of skill in the art.

Definitions as used herein are intended to supplement and illustrate, not preclude, the definitions known to those of skill in the art.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Any patents or publications mentioned in this specification are indicative of the levels of those skilled in the pertinent art. These patents and publications are herein incorporated by reference to the same extent as if each individual publication was specifically and individually indicated to be completely incorporated by reference.

One skilled in the art will readily appreciate that the present disclosure is well-adapted to carry out the objects and obtain the ends and advantages mentioned, as well as those inherent therein. The present examples along with the methods, procedures, treatments, molecules and specific compounds described herein are presently representative of specific aspects, are exemplary, and are not intended as limitations on the scope of any invention, which shall be solely defined by the foregoing claims. It will be apparent that other aspects exist and are encompassed within the spirit of any invention as defined by the scope of the claims.

1. A buffering composition comprising:
one or more buffering agents;
one or more celluloses or derivatives thereof, said celluloses or derivatives thereof at a concentration of less than or equal to 3 wt % of said buffering composition; and
a polyethylene glycol, said polyethylene glycol present at less than or equal to 3 wt % of said buffering composition.

2. The buffering composition of claim 1, wherein said cellulose is a linear or branched alkyl cellulose.

3. The buffering composition of claim 2, wherein said alkyl cellulose is a C₁-C₄ alkyl cellulose.

4. The buffering composition of claim 2, wherein said alkyl cellulose is methyl cellulose, hydroxypropyl cellulose or a combination thereof.

5. The buffering composition of claim 1, wherein said cellulose is present at 0.1 wt % to 3 wt %.

6. The buffering composition of claim 1, wherein said buffering agent is characterized by a pKa of 6-8.

7. The buffering composition of claim 1, wherein said buffering agent comprises a phosphate.

8. The buffering composition of claim 1, wherein said composition further comprises an albumin.

9. The buffering composition of claim 1, wherein said composition comprises 0.1 wt % to 3 wt % methyl cellulose, 0.01 wt % to 3 wt % hydroxypropyl cellulose, and 0.1 wt % to 3 wt % polyethylene glycol.

10. The buffering composition of claim 1, wherein said composition comprises 0.25 wt % to 3 wt % methyl cellulose and 0.25 wt % to 3 wt % hydroxypropyl cellulose.

11. The composition of claim 1, wherein said polyethylene glycol is polyethylene glycol monomethylether 550, poly(ethylene glycol) 8000, or a combination thereof.

12. A method of analyzing a biological sample comprising:
obtaining a biological sample;
forming an analysis composition by contacting said biological sample with a cocktail comprising the buffer composition of claim 1 and one or more antibodies, said one or more antibodies conjugated to one or more florescent dyes; and
subjecting said analysis composition to analysis by detecting one or more of said dyes in contact with a portion of said biological sample.

13. The method of claim 12, wherein said cocktail comprises two or more antibodies specific for different target molecules, wherein a first antibody specific for a first target molecule is conjugated to a first dye, and wherein a second antibody specific for a second target molecule is conjugated to a second dye, said first dye and said second dye characterized by different fluorescent properties.

14. The method of claim 12, comprising 2 to 23 antibodies directed to different target molecules and each conjugated to a different dye.

15. The method of claim 12, wherein at least two of said antibodies are conjugated to different polymer dyes.

16. The method of claim 12, wherein said dyes comprise one or more polymer dyes selected from the group consisting of Brilliant Violet 421™, Brilliant Violet 510™, Brilliant Violet 570™, Brilliant Violet 605™, Brilliant Violet 650™, Brilliant Violet 711™, Brilliant™ Violet 750, or Brilliant Violet 785™.

17. The method of claim 12, wherein said dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.

18. The method of claim 12, wherein said subjecting is by flow cytometry, FISH, immunohistochemistry, sandwich assay, Southern blot, western blot, microarray, or substrate binding assay.

19. A method of forming a cocktail for use in analysis of a biological sample comprising:
combining a buffering composition of claim 1 and one or more antibodies, said one or more antibodies conjugated to one or more florescent dyes.

20. The method of claim 19, wherein said cocktail comprises 0.1 wt % to 3 wt % methyl cellulose, 0.01 wt % to 3 wt % hydroxypropyl cellulose, and 0.1 wt % to about 3 wt % polyethylene glycol.
